(19) United States
(12) Patent Application Publication
(10) Pub. No.: US 2019/0290585 A1
(43) Pub. Date: Sep. 26, 2019

(54) EV-MEDIATED DELIVERY OF BINDING PROTEIN-SMALL MOLECULE CONJUGATES

(71) Applicant: Evox Therapeutics Ltd, Oxford (GB)

(72) Inventor: Oscar WIKLANDER, Solna (SE)

(21) Appl. No.: 16/317,487

(22) PCT Filed: Jul. 11, 2017
(86) PCT No.: PCT/EP2017/067372

§ 371 Date: Jan. 11, 2019

(30) Foreign Application Priority Data
Jul. 12, 2016 (GB) 1612110.5

(51) Int. Cl. A61K 9/127; A61K 31/485; A61P 25/16; A61K 31/506; A61P 35/00; A61P 37/00; A61K 31/438; A61K 38/22; A61P 3/06

(52) U.S. Cl. CPC A61K 9/1277; A61K 31/485; A61P 25/16; A61K 31/506; A61P 3/06; A61P 37/00; A61K 31/438; A61K 38/22; A61P 35/00

(57) ABSTRACT

The present invention relates to extracellular vesicles (EVs) comprising a binding protein which may be used for delivery of protein-drug conjugates comprising the binding protein and a small molecule agent, typically a small molecule drug. The present invention also relates to methods for producing such EVs as well as pharmaceutical compositions and medical uses of such EVs.

### TECHNICAL FIELD

The present invention relates to extracellular vesicles (EVs) comprising a binding protein for delivery of protein-drug conjugates comprising the binding protein and a small molecule agent, typically a small molecule drug.

### BACKGROUND ART

Extracellular vesicles (EVs) modulate cell-to-cell communication in normal physiology and pathology by presenting their contents (primarily RNA, proteins, and lipids) to recipient cells in target tissues. Modification of EVs to incorporate various types of pharmacological agents have been explored in numerous contexts, for instance WO2013/084000, which discloses the use of exosomes for intracellular delivery of biotherapeutics, or WO2010/119256, which describes delivery of exogenous genetic material using exosomes.

The utility of EVs as drug delivery vehicles is unquestionable in the case of for instance nucleic acid based drugs such as siRNA, large protein-based drugs targeting intracellular components, and e.g. poorly soluble or highly toxic small molecule therapeutic agents. EV-mediated small molecule drug delivery has also been explored to a great extent, with for instance WO2011/097480 representing the typical approach to drug loading of EVs. WO2011/097480 describes a very facile method wherein e.g. the phytochemical small molecule agents curcumin and resveratrol are loaded into EVs using a simple co-incubation step during which purified EVs and free drug (e.g. curcumin) are allowed to incubate together in phosphate buffered saline (PBS) at room temperature, relying on diffusion of the drug into the EV. Although highly convenient and straightforward, this conventional approach to loading small molecule agents into EVs is not particularly efficient, results in significant waste of the small molecule agent. Also, the loading process can be very difficult to control. Others (for instance Fuhrman et al, J. Control Rel., 2015) have also evaluated permeabilization of EVs, using detergents such as saponin, as a way of increasing the loading efficiency of in this case the photoactive small molecule agent porphyrin.

A recent patent application (WO2015/120150) is also concerned with loading of tumor-derived EVs with various types of anticancer drugs, covering both small molecules and large biopharmaceuticals. However, as is often the case in the art, very little information is available on how to load exosomes and if there are methods available they are rarely useful for loading and actual therapeutic application of small molecule-carrying EVs.

### SUMMARY OF THE INVENTION

It is hence an object of the present invention to overcome the above-identified problems associated with the loading of EVs with small molecule agents (typically small molecule drugs or diagnostic agents) for subsequent therapeutic application. Furthermore, the present invention aims to satisfy other existing needs within the art, for instance to effectively deliver not only small molecule agents but also conjugates between small molecules and binding proteins present on EVs (herein referred to as binding protein-small molecule conjugates and similar terms) in a targeted and controllable fashion.

The present invention achieves these and other objectives by using EVs as delivery modalities for either small molecule agents as such, or a combination of small molecule agents and binding proteins having a therapeutic and/or prophylactic effect. The EVs as per the present invention may typically comprise a binding protein, which may be a therapeutic protein (such as a receptor), which acts as an interaction partner (i.e. a binder) for at least one small molecule agent (such as a small molecule drug).

Thus, in a first aspect, the present invention relates to EVs comprising a binding protein, characterized in that a small molecule agent is bound to the binding protein. The binder protein may play several different roles: it may for instance be (i) a carrier and/or delivery modality which is primarily meant to transport a small molecule agent attached to it, (ii) a targeting agent to direct trafficking of the EV carrying the binder protein-small molecule conjugate to a particular location, (iii) a therapeutically active protein which becomes therapeutically active or inactive through the attachment of a small molecule, which may have agonistic or antagonistic effects, (iv) a signaling protein which with or without its small molecule cargo may exert or contribute to a cellular and/or bodily change and a related therapeutic and/or prophylactic effect. The binding protein may further contribute to a bodily and/or cellular action or activity and a related therapeutic effect by releasing the small molecule agent in a suitable location, or it may contribute to such effects by retaining the small molecule agent bound to it.

In an advantageous embodiment, the binding protein is a fusion protein comprising the binding protein and an EV protein, in order to enable controlled loading and display of the binding protein onto the EV surface. However, binding proteins naturally occurring in EVs or binding proteins that are engineered to shuttle to EVs are also within the scope of the present invention, and the selection and/or design of the binding protein will be heavily influenced by the disease to be treated, the pharmacological target and the small molecule agent to which the binding protein binds.

In another aspect, the present invention relates to a method of modulating cellular signal transduction, comprising the steps of allowing a small molecule agent to interact non-covalently or covalently with a binding protein displayed on an EV and subsequently exposing the resultant binding protein-small molecule drug conjugate, the binding protein itself, and/or the small molecule agent bound to the binding protein to a particular target and/or a target location.

In yet another aspect, the present invention pertains to methods of producing EVs as per the present invention. Such methods may comprise the steps of (a) providing an EV comprising a binding protein in the form of a fusion protein with an EV protein, and (b) exposing the binding protein to a small molecule agent to enable interaction and binding of the small molecule agent to the binding protein.

Moreover, the invention also relates to methods of delivering a binding protein, a binding protein-small molecule conjugate, and/or a small molecule, comprising the steps of (a) providing an EV according to the invention and (b) delivering the binding protein, the binding protein-small molecule conjugate, and/or the small molecule agent (typically a small molecule drug) to a target location.

Generally, the small molecule agents of the present invention may be selected from a wide variety of drug agents and/or diagnostic agent categories, for instance anticancer agents such as doxorubicin, 5-fluorouracil or other nucleoside analogues such as cytosine arabinoside, proteasome inhibitors such as bortezomib, or kinase inhibitors such as imatinib or seliciclib, or NSAIDs such as naproxen, aspirin, or celecoxib, antibiotics such as heracillin, or antihypertensives such as ACE inhibitors such as enalapril, ARBs such as candesartan. Various types of hetero- or homo-dimeric, trimeric, or multimeric small molecule agents are also within the scope of the present invention, as such agents may facilitate interaction between the binding protein and another protein or non-protein target molecule of interest.

In yet another aspect, the present invention pertains to methods for delivering small molecule drugs to a target location, such as a target cell, a target cellular component such as the cytoplasm or the nucleus, a target tissue, a target organ, or to any target compartment (which may also include bodily fluids, for instance the blood stream or cerebrospinal fluid). Such methods may comprise exposing the target location to EVs loaded with a small molecule, either in the form of a conjugate with a binding protein (a conjugate in the sense that the small molecule is bound to a binding protein) or a small molecule that has been released from a binding protein into the EV or from the EV.

In a further aspect, the present invention also relates to methods of altering the pharmacokinetic or pharmacodynamics profile of a small molecule drug. Such methods comprise loading of the small molecule in question onto a binding protein present on and/or in an EV, in order to modulate in vivo and potentially also in vitro properties of the small molecule drug in question.

Additionally, in further aspect, the present invention pertains to pharmaceutical compositions comprising EVs carrying small molecules in the form of binding protein-small molecule conjugates. In practice, pharmaceutical compositions per the present invention do not merely comprise a small number of EVs but in fact large populations of EVs. The EV concentration in such compositions may be expressed in many different ways, for instance amount of EV protein per unit (often volume) or per dose, number of EVs or particles per unit (often volume, per animal, per kg of body weight, etc.) or per dose, concentration of small molecule drug per unit or per dose, etc. Typically, such pharmaceutical compositions are formulated for in use in vivo and also in vitro using pharmaceutically acceptable excipients.

Finally, the present invention also relates to medical uses and applications of EVs comprising binding protein-small molecule conjugates, for instance in the treatment, diagnosis, prophylaxis or monitoring of inflammatory diseases, autoimmune diseases, cancer, metabolic disorders, or any suitable disease or disorder, or for cosmetic or other non-disease related applications.

### BRIEF DESCRIPTION OF THE DRAWINGS

FIG. 1 shows that EV-loaded APO retains or even increases its function to induce PKA and MAPK dependent signalling that leads to increased production of the downstream pro-survival FGF-2.

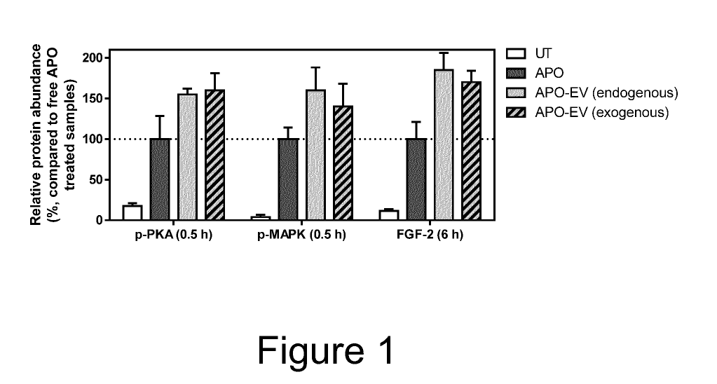

FIG. 2 shows that EVs carrying an ABL1 receptor tyrosine kinase-imatinib conjugate reduce tumour weight and increase survival.

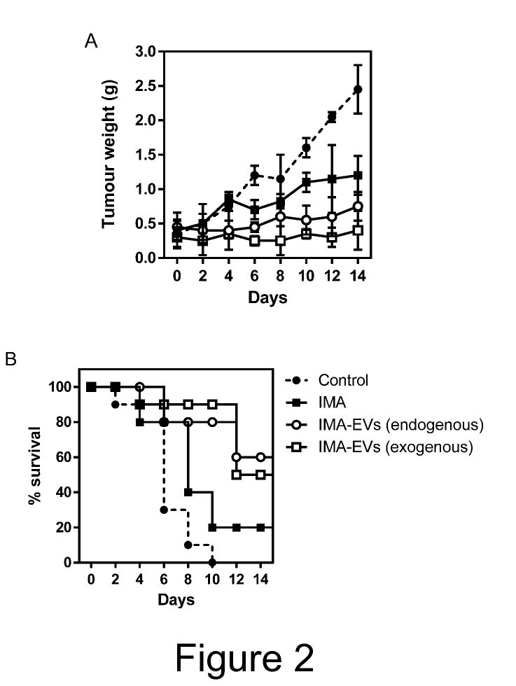

FIG. 3 illustrates the effects of EVs carrying an ABL1 receptor tyrosine kinase-imatinib conjugate in an LPS-induced acute sepsis model.

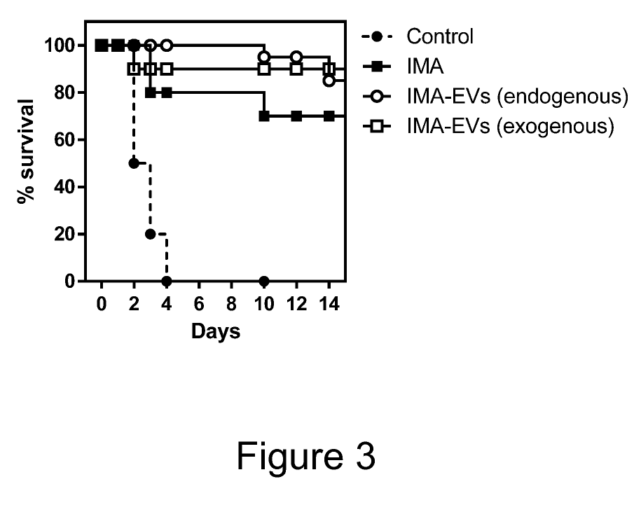

FIG. 4 shows the anticancer effects on MCF7 breast cancer of MSC-derived EVs comprising a binding protein-small molecule conjugate comprising either FKBP12 or a fusion protein comprising FKBP12 and CD63, carrying the small molecule rapamycin which binds to FKBP12.

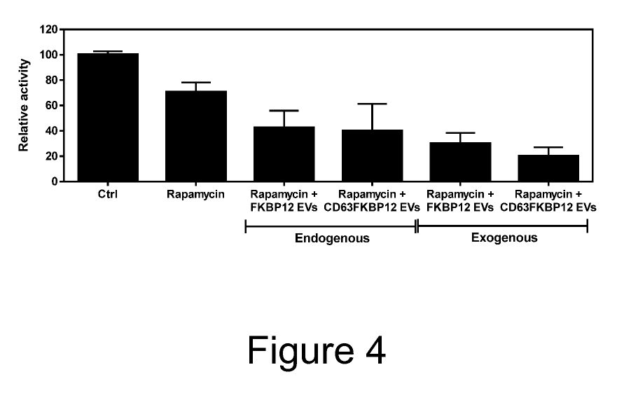

FIG. 5 shows induction of lipolysis in 3T3-L1 cells, using adrenomedullin (AM) loaded onto EVs comprising the binding protein CALCRL (a receptor for AM) or CALCRL fused to the EV protein CD81.

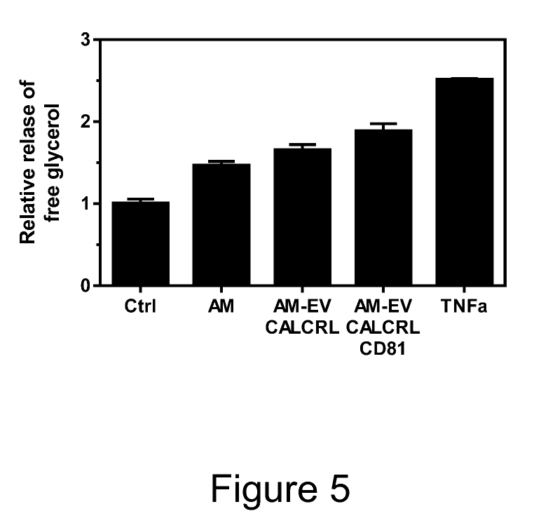

### DETAILED DESCRIPTION OF THE INVENTION

The present invention describes inter alia novel methods, compositions, EVs, and uses of EVs for the delivery of small molecules, protein biologics, and/or protein-small molecule conjugates. Moreover, the present invention relates to methods for EV loading, EVs carrying small molecules, various methods for utilizing such EVs, pharmaceutical compositions comprising EVs in therapeutically effective amounts, and medical uses of small molecule-carrying EVs as per the present invention.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the various small molecules described in connection with the small molecule-carrying EVs are to be understood to be disclosed, relevant and included also in the context of the methods for loading EVs with binding protein-small molecule drug conjugates, that is all small molecule agents and all binding proteins shall be considered disclosing also all of their potential interaction partners Furthermore, certain embodiments described in connection with certain aspects, for instance the administration routes of the small molecule-carrying EVs, as described in relation to aspects pertaining to treating certain medical indications with EVs as such, may naturally also be relevant in connection with other aspects and/or embodiment such as those pertaining to the pharmaceutical compositions of the present invention. Moreover, any and all features (for instance any and all members of a Markush group) may be freely combined with any and all other features (for instance any and all members of any other Markush group), e.g. any binding protein may be combined with any small molecule agent, or any binding protein may be combined with any EV protein, without departing from the gist of the invention Furthermore, when teachings herein refer to EVs in singular and/or to EVs as discrete natural nanoparticle-like vesicles it should be understood that all such teachings are equally relevant for and applicable to a plurality of EVs and populations of EVs. As a general remark, the small molecule agents, the binding proteins, the targeting moieties, the cell sources, the EV proteins, and all other aspects, embodiments, and alternatives in accordance with the present invention may be freely combined in any and all possible combinations without departing from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the technical effect associated therewith. As long as their biological properties are retained the polypeptide and/or polynucleotide sequences according to the present application may deviate with as much as 50% (calculated using for instance BLAST or ClustalW) as compared to the native sequence, although a sequence identity that is as high as possible is preferable (for instance 60%, 70%, 80%, or e.g. 90% or higher). The combination (fusion) of e.g. at least one binder protein and at least one exosomal protein implies that certain segments of the respective polypeptides may be replaced and/or modified, meaning that the deviation from the native sequence may be considerable as long as the key properties (e.g. targeting properties, trafficking to the surface of exosomes, therapeutic activity, binding to a small molecule agent, etc.) are conserved. Similar reasoning thus naturally applies to the polynucleotide sequences encoding for such polypeptides.

The terms “extracellular vesicles” or “EVs” or “exosomes” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall also be understood to relate to lipoprotein particles, such as LDL, VLDL, HDL and chylomicrons, as well as liposomes, hybrid vesicles, extracellular vesicle (EV) mimics, cell membrane-based vesicles obtained through membrane extrusion or other techniques, etc. Essentially, the present invention may relate to any type of lipid-based structure (with vesicular morphology or with any other type of suitable morphology) that can act as a delivery or transport vehicle for small molecules of interest. It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 10¹⁰, 10¹¹, 10¹⁵, 10¹⁸, 10²⁵ EVs (often termed “particles”) per unit of volume (for instance per ml), or any other number larger, smaller or anywhere in between. In the same vein, the term “population”, which may e.g. relate to an EV comprising a certain small molecule, shall be understood to encompass a plurality of essentially similar entities constituting such a population. In other words, individual EVs when present in a plurality constitute an EV population.

Thus, as will be clear to the skilled person, the present invention pertains both to individual EVs comprising small molecules and populations comprising EVs comprising small molecules, normally bound to a binding protein. The dosages of EVs when applied in vivo may naturally vary considerably depending on the disease to be treated, the administration route, the small molecule cargo, etc.

The term “small molecule agent” or “small molecule” or “small molecule drug” or “small molecule therapeutic” are used interchangeably herein and shall be understood to relate to any molecular agent which may be used for the treatment and/or diagnosis of a disease and/or disorder, and also for modulating or changing e.g. the activity and/or the binding and/or the location of a binding protein. Small molecule agents are normally synthesized via chemical synthesis means, but may also be naturally derived, for instance via purification from natural sources, or may be obtained through any other suitable means or combination of techniques. A brief, non-limiting definition of a “small molecule” is any organic compound with a molecular weight of less than 900 g/mol (Dalton) that may in essentially any way regulate, impact, or influence a biological process. For the purposes of this invention, small molecules may be substantially larger than 900 g/mol, for instance 1500 g/mol, 3000 g/mol, or occasionally even larger. Overall, molecular weight and/or molecular size is not a defining factor behind what constitutes a small molecule agent. In fact, for the purposes of the present invention any agent that can be bound by a binding protein displayed on an EV is considered to be a “small molecule agent”. As such, synthetic and natural peptides, RNA-based agents comprising either natural and/or modified nucleotides, and even proteins are considered small molecule agents as per the present invention, as long as they can be bound by the binding protein. In certain embodiments, the small molecule agent may be an oligonucleotide such as a short interfering RNA (siRNA), a short hairpin RNA (shRNA), a CRISPR guide RNA strand, an mRNA, an antisense oligonucleotide, or a splice-switching oligonucleotide, or any other types of RNA molecules. In yet another embodiment, the small molecule agent may be a peptide such as a receptor ligand, a neuropeptide, an Aβ inhibitor, a cell-penetrating peptide (CPP), a peptide inducing endosomal escape, a targeting peptide, or any other suitable peptide. Although small molecules often exhibit good oral bioavailability many small molecule drugs need to be given intravenously or via some other route of administration, be it for pharmacokinetic, pharmacodynamics, and/or toxicity or stability reasons. Examples of small molecules include anticancer agents such as doxorubicin, daunorubicin, 5-fluorouracil, methotrexate, proteasome inhibitors such as bortezomib, or kinase inhibitors such as imatinib or seliciclib, NSAIDs such as naproxen, aspirin, or celecoxib, antibiotics such as heracillin, antihypertensives such as ACE inhibitors such as enalapril, ARBs such as candesartan, oligonucleotides such as siRNA, splice-switching RNA, peptides, heterodimeric or homodimeric small molecules, etc. The present invention is naturally applicable also to essentially any other small molecule without departing from the gist of the invention, as would be clear to a person skilled in the art.

The terms “binder protein, “binding protein”, “binder”, “receptor protein” and similar terms are used interchangeably herein and shall be understood to relate to any protein, polypeptide, or peptide (i.e. any molecule comprising a sequence of amino acids) to which a small molecule agent can be attached, via non-covalent or covalent attachment, or via a combination of both covalent and non-covalent interactions. The combination of a binding protein and a small molecule agent is herein described using terms such as “binding protein-small molecule conjugate” or “binding protein-small molecule drug conjugate” or “binding protein-small molecule agent conjugate”, or just “conjugate”. The binding protein may play several different roles: it may for instance be (i) a carrier and/or delivery modality which is primarily meant to transport a small molecule agent attached to it, (ii) a targeting agent to direct trafficking of the EV carrying the binder protein-small molecule conjugate to a particular location, (iii) a therapeutically active protein which becomes therapeutically active or inactive through the attachment of a small molecule, which may have agonistic or antagonistic effects, (iv) a signaling protein which together with or without its small molecule cargo may exert or contribute to a cellular and/or bodily change and a related therapeutic and/or prophylactic effect, (v) a protein carrying out or catalyzing a particular reaction only when brought into the proximity of another protein, etc. The binding protein may further contribute to a bodily and/or cellular action or activity and a related therapeutic effect by releasing the small molecule agent in a suitable location, or it may contribute to such effects by retaining the small molecule agent bound to it. An example of the first case is when the binding protein releases the small molecule drug inside a target cell after EV-mediated delivery, whereas an example of the second case is the delivery of an antibody-small molecule drug conjugate into a tumor. Typically, the binding protein is of human origin, although in certain instances known to a skilled person the binding protein may be obtainable from any other species (such as, using a non-limiting example, when the binding protein is a nuclease like Cas9, which is a bacterial protein). Furthermore, the binding protein does not need to be present in its entirety but may be present in the form of a subunit, a domain, a truncated protein, a derivative or a variant thereof, as long as the desired effect (be it an effect related to delivery, to therapeutic activity, to targeting, etc.) is maintained. Binding proteins may be naturally occurring in EVs and/or in EV source cells, or they may be trafficked to EVs via fusions constructs with EV proteins. Non-limiting examples of binding proteins as per the present invention includes GPCRs, polyclonal and monoclonal antibodies, single chain variable fragments (scFv), integrins, enzymes such as tyrosine kinases (for instance BTK or Bcr-Abl tyrosine kinase), nucleases such as Cas and Cas9, proteases, integrases, phosphatases, ligases, GTPases, DNA-binding and RNA-binding proteins such as Ago2, Dicer, GW182, hnRNPA1, hnRNPA2B1, DDX4, ADAD1, DAZL, ELAVL4, IGF2BP3, SAMD4A, TDP43, FUS, FMR1, FXR1, FXR2, EIF4A1-3, the MS2 coat protein; aromatases or esterases, adaptor proteins (such as an SH2 domain), G-proteins, GEFs, calmodulin, Ras, talin, vinculin, paxilin, Raf, caspases, transcription factors such as MyoD and Myf5, tumor suppressors such as p53, neurotrophic factors such as brain-derived neurotrophic factor (BDNF) and nerve growth factor, neurotransmitter receptors, dopamine receptors, structural proteins such as dystrophin, utrophin, titin, nebulin, dystrophin-associated proteins such as dystrobrevin, syntrophin, syncoilin, desmin, sarcoglycan, dystroglycan, sarcospan, agrin, and/or fukutin, interleukins such as IL1 alpha and beta, IL2, IL4, IL6, IL10, IL17, IL23, and other interleukins and all interleukin receptors such as IL2R, IL6R, gp130, IL10R, IL17R, IL23R, interferons (such as INF alpha and beta) and interferon receptors, tumor necrosis factors (TNFs) such as TNF alpha and beta and their receptors, annexins such as ANXA2 and ANXAS, signal recognition particle and receptor components such as SRPB2 and SRP54, extracellular matrix components such as COL1A1 and COL6A1, solute carriers SLC25A5 and SLC25A13, ribosomal proteins RPS27A and RPL7, translation elongation factors such as EEF1A1 and EEF1A2, initiation factors such as EIF4A1 and EIF4A2, GPI-anchored proteins, tetraspanins such as CD63 and CD81, tissue factor, growth factors and their receptors such as EGF and EGFR, and any fusions, combinations, subunits, derivatives, or domains of any of the above binding proteins.

The terms “EV protein”, “exosomal protein”, “exosomal sorting domain”, “EV sorting domain”, “EV sorting protein”, “exosomal protein”, “exosomal polypeptide”, “EV polypeptide”, etc. are used interchangeably herein and shall be understood to relate to any polypeptide that can be utilized to transport a polypeptide construct (which typically comprises, in addition to the EV protein, at least one protein of interest) to a suitable vesicular structure, i.e. to a suitable EV. More specifically, said terms shall be understood as comprising any polypeptide that enables transporting, trafficking or shuttling of a polypeptide construct to a vesicular structure, such as an exosome. Examples of such exosomal sorting domains are for instance CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof, but numerous other polypeptides capable of transporting a polypeptide construct to an EV are comprised within the scope of the present invention. The EV proteins as per the present invention are typically of human origin and can be found in various publicly available databases such as Uniprot, RCSB, etc. The EV proteins may be fused to various other proteins and/or protein domains, to for instance enhance the surface display, increase avidity, or enable interaction with particular types of binding proteins in a non-covalent manner.

The terms “source cell” or “EV source cell” or “parental cell” or “cell source” or “EV-producing cell” or any other similar terminology shall be understood to relate to any type of cell that is capable of producing EVs, e.g. exosomes, under suitable cell culturing conditions. Such conditions may be suspension cell culture or adherent culture or any other type of culturing system. Hollow-fiber bioreactors, stir-tank bioreactors and other types of bioreactors represent highly suitable cell culturing infrastructure. The source cells per the present invention may be select from a wide range of cells and cell lines, for instance mesenchymal stem or stromal cells or fibroblasts (obtainable from e.g. bone marrow, adipose tissue, Wharton's jelly, perinatal tissue, tooth buds, umbilical cord blood, skin tissue, etc.), amnion cells and more specifically amnion epithelial cells optionally expressing various early markers, myeloid suppressor cells. Cell lines of particular interest include human umbilical cord endothelial cells (HUVECs), human embryonic kidney (HEK) cells, endothelial cell lines such as microvascular or lymphatic endothelial cells, chondrocytes, MSCs, airway or alveolar epithelial cells, and various other non-limiting examples of cell sources.

The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a logistical standpoint, as such sources provide for off-the-shelf therapies

In a first aspect, the present invention relates to an EV comprising a binding protein, wherein a small molecule agent (for instance, a small molecule drug) is bound to the binding protein. Typically, the small molecule agent is bound to the binding protein through non-covalent attachment/linkages, but the attachment may also be based on covalent interaction(s). A non-limiting example of a non-covalent interaction is the attachment between an ABL1 receptor tyrosine kinase and the small molecule drug imatinib. A non-limiting example of a covalent interaction between a binding protein and a small molecule drug is the covalent bond which typically connects a monoclonal antibody and an anticancer agent in an antibody-drug conjugate, for instance the covalent linkage between brentuximab and monomethyl auristatin E (brentuximab-vedotin). Although advantageous, release of the small molecule drug from the binding protein is not a requirement, as long as the small molecule drug, the binding protein, and/or the protein-drug conjugate can exert its desired effect. Suitable non-limiting examples of covalent bonds that are releasable include disulfide bridges and thioether bonds, which may undergo reduction in reductive environments, amide bonds which may be cleaved by e.g. proteases and other enzymes, biotin-streptavidin linkages which dissociate under certain in vivo conditions, etc. There are nonetheless multiple strategies available for covalent conjugation/linkage of a small molecule drug to the binding protein, with non-limiting examples such as an ester bond, an amide bond, a disulfide bond, a thioether bond, a biotin-streptavidin interaction, a linkage obtained through a maleimide-NHS reaction, a linkage obtained through a EDC-NHS reaction, a stapled linkage (for instance an all-hydrocarbon staple) and various other bonds.

In an advantageous embodiment, the binding protein is a fusion protein comprising the binding protein per se fused to an EV protein. As above-mentioned, the binding protein does not necessarily have to be a protein in its entirety but may be a sub-unit, a domain, a derivative, a truncation, or any other type of modified sequence of amino acids as compared to the native sequence. One example of such a modification is the use of a single chain fragment variable (scFv) domain of an antibody, instead of using the antibody in its entirety.

The binding proteins as per the present invention may be essentially any protein that can bind a small molecule as per the present invention. As above-mentioned, some non-limiting examples of binding proteins as per the present invention includes GPCRs, polyclonal and monoclonal antibodies, single chain variable fragments (scFv), integrins, enzymes such as tyrosine kinases, nucleases such as Cas and Cas9, DNA-binding or RNA-binding proteins or domains thereof, proteases, ligases, isomerases, integrases, phosphatases, GTPases, aromatases or esterases, adaptor proteins such as an SH2 domain, G-proteins, GEFs, calcium-binding proteins such as calmodulin, Ras, talin, vinculin, paxilin, Raf, caspases, transcription factors such as MyoD and Myf5, tumor suppressors such as p53, p21, pVHL, APC, CD95, ST5, YPEL3, ST7, and ST14, neurotrophic factors such as brain-derived neurotrophic factor (BDNF) and nerve growth factor, structural proteins such as Dystrophin, utrophin, titin, nebulin, dystrophin-associated proteins such as dystrobrevin, syntrophin, syncoilin, desmin, sarcoglycan, dystroglycan, sarcospan, agrin, and/or fukutin, and any fusions, combinations, subunits, derivatives, or domains of any of the above binding proteins.

Binding proteins in the form of antibodies may in non-limiting embodiments include any one or more of Abagovomab, Abciximab, Actoxumab, Adalimumab, Adecatumumab, Aducanumab, Afelimomab, Afutuzumab, Alacizumab Alemtuzumab, Alirocumab, Altumomab pentetate, Amatuximab, Anatumomab mafenatox, Anifrolumab, Anrukinzumab, Apolizumab, Arcitumomab, Aselizumab, Atinumab, Atlizumab, Atorolimuma, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, cBR96-doxorubicin immunoconjugate, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, Ticilimumab, Tildrakizumab, Tigatuzumab, Tocilizumab, Toralizumab, Tositumomab, Bexxar, Tovetumab, Tralokinumab, Trastuzumab, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, Zolimomab aritox, or any combination thereof. Antibodies may play several roles when utilized as binding proteins. For instance, antibodies may (i) provide for targeted delivery to cell, tissues, and/or organs of interest, (ii) have inherent therapeutic activity, (iii) when attached to EV surfaces, they may be delivered into target cells in vitro and in vivo, (iv) act as binding proteins aimed to deliver small molecule drugs (often referred to as antibody-drug conjugates (ADCs) into cells or extracellularly in the body), and (v) provide several binding sites for a plurality of small molecule agents.

As abovementioned, the small molecule drugs as per the present invention may be obtained from essentially the entire space of pharmaceutically and/or pharmacologically and/or diagnostically relevant agents, for instance anticancer agents, cytostatic agents, tyrosine kinase inhibitors, statins, NSAIDs, antibiotics, antifungal agents, antibacterial agents, antiparasitics, anti-inflammatory agents, anti-fibrotics, antihypertensives, aromatase or esterase inhibitors, an anticholinergics, SSRIs, BKT inhibitors, PPAR agonists, HER inhibitors, AKT inhibitors, BCR-ABL inhibitors, signal transduction inhibitors, angiogenesis inhibitors, synthase inhibitors, ALK inhibitors, BRAF inhibitors, MEK inhibitors, PI3K inhibitors, neprilysin inhibitors, beta2-agonists, CRTH2 antagonists, FXR agonists, BACE inhibitors, sphingosine-1-phosphate receptor modulators, MAPK inhibitors, Hedgehog signaling inhibitors, MDM2 antagonists, LSD1 inhibitors, lactamase inhibitors, TLR agonists, TLR antagonists, IDO inhibitors, ERK inhibitors, Chk1 inhibitors, splicing modulatory, DNA or RNA intercalators, vasodilators, monomeric, dimeric, trimeric, and/or multimeric small molecules that are either heteromeric or homomeric, etc. Other non-limiting examples of small molecule drugs as per the present invention includes for instance everolimus, trabectedin, abraxane, pazopanib, enzastaurin, vandetanib, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, pemetrexed, erlotinib, dasatanib, nilotinib, decatanib, panitumumab, amrubicin, oregovomab, nolatrexed, batabulin, ofatumumab, zanolimumab, edotecarin, tetrandrine, rubitecan, tesmilifene, oblimersen, ticilimumab, ipilimumab, gossypol, cilengitide, gimatecan, lucanthone, neuradiab, vitespan, talampanel, atrasentan, romidepsin, sunitinib, 5-fluorouracil, vorinostat, etoposide, gemcitabine, doxorubicin, 5′-deoxy-5-fluorouridine, vincristine, temozolomide, seliciclib, capecitabine, camptothecin, PEG-labeled irinotecan, tamoxifen, toremifene citrate, anastrazole, exemestane, letrozole, vatalanib, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, erlotinib, lapatanib, canertinib, lonafarnib, tipifarnib, amifostine, suberoyl analide hydroxamic acid, valproic acid, trichostatin sorafenib, arnsacrine, anagrelide, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, squalamine, endostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, droloxifene, 4-hydroxytamoxifen, pipendoxifene, arzoxifene, fulvestrant, acolbifene, lasofoxifene, idoxifene, topotecan, rapamycin, temsirolimus, zolendronate, prednisone, lenalidomide, gemtuzumab, hydrocortisone, dexrazoxane, alemtuzumab, all-transretinoic acid, ketoconazole, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, efavirinz among others. As mentioned above, the present invention is naturally applicable also to other small molecules without departing from the gist of the invention, as would be clear to a person skilled in the art.

As above-mentioned, the binding protein is advantageously expressed on the EV as a fusion protein between the binding protein per se and a suitable EV protein. The EV protein may be selected from the group comprising proteins such as CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof, but numerous other polypeptides capable of transporting a polypeptide construct to an EV are comprised within the scope of the present invention. The EV proteins are typically of human origin and can be found in various publicly available databases such as Uniprot, RCSB, etc. The EV proteins may be fused to various other proteins and/or protein domains, to for instance enhance the surface display, increase avidity, or enable interaction with particular types of binding proteins in a non-covalent manner.

In a further embodiment, the binding of the small molecule agent may make the binding protein therapeutically active. A non-limiting example of this may be the binding to the binding protein by an agonistic or an antagonistic small molecule, which may render the binding protein therapeutically activated, for instance through enabling signaling or through inhibiting signaling. Thus, in this particular case the binding protein may become therapeutically activated when signaling-competent, but signaling-incompetent binding proteins may also be therapeutically active, for instance it may be able to carry out a pharmacological effect via impeding a particular signaling pathway. Yet another non-limiting example of therapeutically activated binding proteins are enzymes, wherein the enzymatic activity is optionally requiring the presence of both the small molecule agent and a target.

In yet another embodiment, the EVs as per the present invention may comprise at least one targeting moiety displayed on the surface of the EV, to even further enhance its therapeutic potential by targeting a tissue, an organ, or cell type of interest. The targeting moiety normally comprises a sequence of amino acids, which may be identified for instance through phage display or any other type of screening methodology. The targeting moiety is typically displayed on the EV surface through genetic engineering of the EV source cells, wherein the source cells are transfected to produce EVs comprising a fusion protein comprising the targeting moiety and an exosomal protein. EV proteins as per the present invention includes, inter alia, CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof, but numerous other polypeptides capable of transporting a polypeptide construct to an EV are comprised within the scope of the present invention. The EV proteins are typically of human origin and can be found in various publicly available databases such as Uniprot, RCSB, etc.

In a further aspect, the present invention relates to a method of delivering to a target location at least one of (i) a small molecule drug, (ii) the binding protein in a therapeutically active form or in a form that is ready for therapeutic activation when in contact with e.g. a target, and/or (iii) a binder protein-small molecule drug conjugate. Such delivery methods may comprise exposing a target cell, a target tissue, or target organ (which may include fluids and liquids such as blood, bile, lymph, interstitial fluid, cerebrospinal fluid, etc.) to EVs as per the present invention. As above-mentioned, the EVs may comprise a targeting moiety expressed on its surface, or it may rely on natural tropism and targeting, or it may be non-targeted. Delivery to a target cell and into a target cell can be carried out in vitro and/or in vivo, depending on the context. Further, the present invention pertains to a method of altering the pharmacokinetic or pharmacodynamics profile of a small molecule drug, or of a protein-drug conjugate. This is achieved through loading the small molecule agent and the binding protein in question into and onto an EV, which will affect factors such as adsorption, distribution, metabolism, enzymatic activity, tissue penetration, clearance, etc.

In yet another aspect, the present invention relates to methods of modulating cellular signal transduction, comprising the steps of binding a small molecule agent to a binding protein displayed on an EV and exposing the conjugate comprising the binding protein and the small molecule agent to a target signaling pathway.

In a further aspect, the present invention pertains to methods of producing EVs as per the present invention. Such methods may be carried out using EVs obtained from any suitable EV-producing cell, for instance MSCs, fibroblasts, immune cells, HEK cells, or any other suitable cell type. So called exogenous methods for producing EVs comprising binding protein-drug conjugates may comprise the steps of (a) providing an EV comprising a binding protein in the form of a fusion protein with an EV protein, and (b) exposing the binding protein to a small molecule agent, to enable non-covalent or covalent binding of the small molecule agent to the binding protein. Normally, the above steps may be preceded by genetically modifying the EV-producing cells to secrete EVs having displayed on their surfaces binding proteins, optionally in the form of fusion proteins with EV polypeptides, such as CD63, CB81, CD9, or Lamp2b or any other suitable EV polypeptide. After secretion of EVs, the culture medium which is conditioned with EVs (i.e. contain EVs) may be purified using a variety of methods as will be described in more detail below, followed by exposure to a small molecule drug of interest. Exposure to the small molecule drug will enable interaction between the drug in question and the binding protein, leading to the formation of a linkage and thus the creation of a binder protein-small molecule drug conjugate.

In a further aspect, the methods of producing EVs as per the present invention may also comprise endogenous loading of EV binding proteins with small molecule drugs. Such methods may comprise steps such as (a) incubating a small molecule agent with a culture of EV source cells comprising a binding protein, optionally in the form of a fusion protein with an EV protein. Subsequently, EVs produced by the EV source cells are harvested, wherein the EVs comprise a small molecule agent bound to the binding protein.

In some embodiments, the methods of producing EVs comprising binding protein-small molecule conjugates may comprise the steps of: genetically modifying EV-producing cells to secrete EVs comprising adaptor proteins. The adaptor proteins, which optionally may be fused to EV proteins, can be used to non-covalent attach various types of binding proteins, for instance antibodies. Thus, in one non-limiting example, after production of the EVs comprising the adaptor proteins, a first exogenous loading step may be carried out, during which e.g. antibodies are bound to the EVs. Subsequently, a second exogenous loading step may take place, wherein a small molecule agent may be attached to the antibody or any other binding protein. Optionally, these two loading steps can be carried out as one single step, by coating the EV with the binding protein which is already pre-loaded with the small molecule agent.

In yet another aspect, the present invention pertains to pharmaceutical compositions comprising EVs comprising binding protein-small molecule conjugates. Typically, the pharmaceutical compositions as per the present invention comprise one type of therapeutic EV (i.e. a population of EVs comprising a certain desired small molecule(s) combined with its binding protein partner) formulated with at least one pharmaceutically acceptable excipient, but more than one type of EV population may be comprised in a pharmaceutical composition, for instance in cases where a combinatorial treatment is desirable. The at least one pharmaceutically acceptable excipient may be selected from the group comprising any pharmaceutically acceptable material, composition or vehicle, for instance a solid or liquid filler, a diluent, an excipient, a carrier, a solvent or an encapsulating material, which may be involved in e.g. suspending, maintaining the activity of or carrying or transporting the EV population from one organ, or portion of the body, to another organ, or portion of the body (e.g. from the blood to any tissue and/or organ and/or body part of interest).

The present invention also relates to cosmetic and dermatological applications of binding protein-small molecule-carrying EVs. Thus, the present invention pertains to skin care products such as creams, lotions, gels, emulsions, ointments, pastes, powders, liniments, sunscreens, shampoos, etc., comprising a suitable EV, in order to improve and/or alleviate symptoms and problems such as dry skin, wrinkles, folds, ridges, and/or skin creases. In one embodiment, EVs (which comprise a small molecule of interest) are obtained from a suitable EV-producing cell source with regenerative properties (for instance a mesenchymal stem cell) are comprised in a cosmetic cream, lotion, or gel for use in the cosmetic or therapeutic alleviation of wrinkles, lines, folds, ridges and/or skin creases.

In yet another aspect, the present invention relates to EVs as per the present invention for use in medicine. Naturally, when an EV comprising a small molecule in accordance with the present invention is used in medicine, it is in fact normally a population of EVs that is being used. The dose of EVs administered to a patient will depend on the amount small molecule drug that has been loaded into the EV, the disease or the symptoms to be treated or alleviated, the administration route, the pharmacological action of the small molecule itself, the inherent properties of the EV, as well as various other parameters of relevance. The EVs and the EV populations thereof as per the present invention may thus be used for prophylactic and/or therapeutic purposes, e.g. for use in the prophylaxis and/or treatment and/or alleviation of various diseases and disorders. A non-limiting sample of diseases wherein the EVs as per the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, non-alcoholic steatohepatitis (NASH), non-alcoholic fatty liver disease (NAFLD), fibrosis, Guillain-Barré syndrome, acute myocardial infarction, ARDS, sepsis, meningitis, encephalitis, liver failure, kidney failure, heart failure or any acute or chronic organ failure and the associated underlying etiology, graft-vs-host disease, Duchenne muscular dystrophy, Becker muscular dystrophy, and other muscular dystrophies, lysosomal storage diseases such as Gaucher disease, Fabry's disease, MPS I, II (Hunter syndrome), and III, Niemann-Pick disease, cystinosis, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The binding protein-small molecule EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the small molecule drug or the EV population as such.

The methods of the present invention may also comprise exposing the EV source cells to serum starvation, hypoxia, bafilomycin, or cytokines such as TNF-alpha and/or IFN-gamma, in order to influence the yield or properties of the resulting EVs. The EV production scale and timeline will be heavily dependent on the EV-producing cell or cell line and may thus be adapted accordingly by a person skilled in the art. The methods as per the present invention may further comprise an EV purification step, wherein the EVs are purified through a procedure selected from the group of techniques comprising liquid chromatography (LC), high-performance liquid chromatography (HPLC), spin filtration, tangential flow filtration, hollow fiber filtration, centrifugation, immunoprecipitation, flow field fractionation, dialysis, microfluidic-based separation, etc., or any combination thereof. In an advantageous embodiment, the purification of the EVs is carried out using a sequential combination of filtration (preferably ultrafiltration (UF), tangential flow filtration or hollow fiber filtration) and size exclusion liquid chromatography (LC). This combination of purification steps results in optimized purification, which in turn leads to superior therapeutic activity. Further, as compared to ultracentrifugation (UC), which is routinely employed for purifying exosomes, sequential filtration-chromatography is considerably faster and possible to scale to higher manufacturing volumes, which is a significant drawback of the current UC methodology that dominates the prior art. Another advantageous purification methodology is tangential flow filtration (TFF), which offers scalability and purity, and may be combined with other types of purification techniques such as filtration.

It shall be understood that the above described exemplifying aspects, embodiments, alternatives, and variants can be modified without departing from the scope of the invention. The invention will now be further exemplified with the enclosed examples, which naturally also can be modified considerably without departing from the scope and the gist of the invention.

### Materials and Methods

Various fusion proteins comprising a binding protein and an EV protein (such as CD81, CD63, CD9, syntenin, syndecan, Alix, CD133, etc.) have been designed and assessed. ORFs were typically generated by synthesis and cloned into the mammalian expression vector pSF-CAG-Amp. Briefly, synthesized DNA and vector plasmid were digested with enzymes NotI and SalI as per manufacturers instruction (NEB). Restricted, purified DNA fragments were ligated together using T4 ligase as per manufacturer's instruction (NEB). Successful ligation events were selected for by bacterial transformation on ampicillin-supplemented plates. Plasmid for transfection was generated by ‘maxi-prep’, as per manufacturer's instruction.

Most of the cloning was performed using the NEBuilder HiFi DNA Assembly Cloning Kit (NEB, Inc.) and confirmed using Sanger sequencing (Source BioScience). Plasmids were transformed into the NEB 5-alpha Competent E. coli cells (NEB, Inc.) and grown overnight in a shaking incubator according to manufacturer's recommendations. Plasmids were isolated and purified using the ‘maxi-prep’ kit, as per manufacturer's instruction (Macherey-Nagel).

Depending on the experimental design and assays, in certain cases, non-viral transient transfection and exosome production was carried out in conventional 2D cell culture, whereas in other cases virus-mediated transduction was employed to create stable cell lines, which were typically cultured in bioreactors of different types such as hollow-fiber bioreactors and stir-tank bioreactors. For conciseness, only a few examples are mentioned herein.

HEK293T cells were typically seeded into 15 cm dishes (9×10⁶ cells per dish) and left overnight in serum-containing DMEM as recommended by ATCC. The following day the cells were transiently transfected with lipoplexed DNA added directly onto cells. Briefly, DNA and polyethyleneimine (PEI) were separately incubated in OptiMEM for 5 minutes before combining together for 20 minutes at room temperature. Lipoplexed DNA and cells were co-incubated for 6 hours following which conditioned culture media was changed to OptiMEM for 48 hours. Other cells and cell lines that were evaluated in dishes, flasks and other cell culture vessels included bone marrow-derived mesenchymal stromal cells (BM-MSCs) and Wharton's jelly-derived MSCs (WJ-MSCs), amnion cells, fibroblasts, various endothelial and epithelial cells, as well as various immune cells and cell lines.

In the case of viral transduction and creation of stable cell lines, cell sources such as BM-MSCs, WJ-MSC, fibroblasts, amnion cells, fibroblasts, various endothelial and epithelial cells, were virus-transduced, typically using lentivirus (LV). Typically, 24 hours before infection, 100.000 cells (e.g. fibroblasts, MSCs, etc.) or 200.000 cells (e.g. HEK293T) are plated in a 6-well plate. 2 uL of LV and optionally Polybrene (or hexadimethrine bromide, final concentration on the well of 8 ug/mL) are added, and 24 hours post transduction the cell medium of transduced cells is changed to fresh complete media. At 72 hours post transduction, puromycin selection (4-6 μg/ml) is performed, normally for 7 days followed by analysis of stable expression of the polypeptide construct.

Stable cells were cultured in either 2D culture or in bioreactors, typically hollow-fiber bioreactors and stir-tank bioreactors, and conditioned media was subsequently harvested for exosome preparation. Various preparation and purification steps were carried out. The standard workflow comprises the steps of pre-clearing of the supernatant, filtration-based concentration, chromatography-based removal of protein contaminants, and optional formulation of the resultant exosome composition in a suitable buffer for in vitro and/or in vivo assays.

As for assays and analytics, Western blot was used to evaluate the enrichment of binding proteins, optionally fused to exosomal proteins, in EVs. Briefly, SDS-PAGE was performed according to manufacturer's instruction (Invitrogen, Novex PAGE 4-12% gels), whereby 1×10¹⁰ exosomes and 20 ug cell lysate were loaded per well. Proteins from the SDS-PAGE gel were transferred to PVDF membrane according to manufacturer's instruction (Immobilon, Invitrogen). Membranes were blocked in Odyssey blocking buffer (Licor) and probed with antibodies against the binding protein and the exosomal protein according to supplier's instruction (Primary antibodies—Abcam, Secondary antibodies—Licor). Molecular probes visualized at 680 and 800 nm wavelengths.

For EV size determination, nanoparticle tracking analysis (NTA) was performed with a NanoSight instrument equipped with analytical software. For all recordings, we used a camera level of 13 or 15 and automatic function for all post-acquisition settings. Electron microscopy and fluorescence microscopy were frequently used to quantitate and analyze EVs.

EVs were isolated and purified using a variety of methods, typically a combination of filtration such as TFF and liquid chromatography. Typically, EV-containing media was collected and subjected to a low speed spin at 300 g for 5 minutes, followed by 2000 g spin for 10 minutes to remove larger particles and cell debris. The supernatant was then filtered with a 0.22 μm syringe filter and subjected to different purification steps. Large volumes were diafiltrated and concentrated to roughly 20 ml using the Vivaflow 50R tangential flow (TFF) device (Sartorius) with 100 kDa cutoff filters or the KR2i TFF system (SpectrumLabs) with 100 or 300 kDa cutoff hollow fibre filters. The preconcentrated medium was subsequently loaded onto the bead-eluate columns (HiScreen or HiTrap Capto Core 700 column, GE Healthcare Life Sciences), connected to an AKTAprime plus or AKTA Pure 25 chromatography system (GE Healthcare Life Sciences). Flow rate settings for column equilibration, sample loading and column cleaning in place procedure were chosen according to the manufacturer's instructions. The sample was collected according to the UV absorbance chromatogram and concentrated using an Amicon Ultra-15 10 kDa molecular weight cut-off spin-filter (Millipore) to a final volume of 100 μl and stored at −80° C. for further downstream analysis. To assess the protein and RNA elution profiles, media was concentrated and diafiltrated with KR2i TFF system using 100 kDa and 300 kDa hollow fibre filters and a sample analysed on a Tricorn 10/300 Sepharose 4 Fast Flow (S4FF) column (GE Healthcare Life Sciences). Exogenous loading of small molecule drugs onto the EVs comprising the binding protein was carried out by co-incubation for suitable time periods, followed by re-purification using essentially the same steps or at least parts of these steps.

### Example 1: Apomorphine Loaded EVs for Parkinson's Disease

MSCs endogenously expressing dopamine receptor D2, and MSCs genetically engineered to express a fusion protein comprising the EV protein CD63 and dopamine receptor D2, were cultured in MSCGM growth medium. To endogenously load MSC-EVs with apomorphine (APO), a drug used for the therapy of Parkinson's disease, MSC culture medium was replaced with Opti-MEM medium and incubated with 2 μM APO for 12 h. Thereafter, the conditioned medium was collected and APO-EVs isolated via tangential flow filtration and size exclusion chromatography. To exogenously load MSC-EVs with APO, EVs from untreated MSCs were isolated using as above, then incubated with 2 μM APO for 2 h, and re-isolated to remove APO molecules that had not been loaded to EVs. APO loading to EVs is facilitated by its interaction of dopamine receptor D2 on EV surface which leads to “precharging” of the signalling competent dopamine receptor.

The activity of APO-EVs, obtained from both genetically modified and unmodified MSCs, was tested to enhance dopamine receptor signalling in primary astrocytes. Astrocytes were cultured in serum free DMEM/F12 medium and treated with 2 μM free APO, or APO-EVs (loaded either endogenously or exogenously) for 0.5 or 6 h. Signalling activity was assessed by measuring the levels of phosphorylated protein kinase A (p-PKA) and phosphorylated mitogen-activated protein kinases (p-MAPK) in astrocyte cell lysate at the 0.5 h time point, and the downstream production of fibroblast growth factor 2 (FGF-2) was assessed in astrocyte conditioned medium at the 6 h time point. The level of p-PKA, p-MAPK and FGF-2 was assessed by semi-quantitative Western blotting using the LI-COR imaging system.

### Example 2: Imatinib Loading to EVs for Cancer Therapy

MSCs endogenously expressing ABL1 receptor tyrosine kinase and MSCs genetically engineered to express a fusion protein of the EV protein syntenin and ABL1 receptor tyrosine kinase were cultured in MSCGM growth medium. To endogenously load MSC-EVs with imatinib (IMA), MSC culture medium was replaced with Opti-MEM medium and incubated with 2 mg IMA. Thereafter, the conditioned medium was collected and IMA-EVs isolated via tangential flow filtration and size exclusion chromatography. To exogenously load MSC-EVs with IMA, EVs from untreated MSCs were isolated using as above, then incubated with 2 mg IMA for 2 h, and re-isolated to remove IMA molecules that had not been loaded to EVs.

IMA is a drug used for the therapy of chronic myelogenous leukaemia (CML) due to its binding to ABL1 and subsequent stabilisation of the Bcr-Abl receptor tyrosine kinase complex, rendering IMA an efficient inhibitor of the kinase. IMA loading to EVs is facilitated by its interaction with ABL1 sorted to EVs, either endogenously or with the aid of an EV protein such as syntenin.

The activity of IMA-EVs was assessed in nude mice after inoculating mice with KU812 cell xenografts and measuring tumour weight and mouse survival after i.p. treatment with free IMA or IMA-EVs in equivalent daily dose of 50 mg/kg IMA.

### Example 3: Imatinib Loading to EVs for Preventing Intravenous LPS-Induced Sepsis

Cell culture, EV loading and EV isolation was performed as in Example 2, but the activity of EV loaded IMA was tested in intravenous LPS induced acute sepsis model. It is known that LPS treatment induces high level of reactive oxygen species that are implicated in septic shock and ARDS. IMA function to increase catalase activity has been related to lowered DNA damage and lowered production of pro-inflammatory cytokines TNF-alpha and IL-6.

Mice were sensitised to LPS by retro-orbital injection of 5 mg/kg of LPS. IMA and EV-IMA treatment was started one day before conducted daily, with equivalent of 200 mg/kg/day IMA, and mouse survival monitored.

### Example 4. Rapamycin Loading to EVs for Inhibiting mTOR Activity

MSCs endogenously expressing peptidyl-prolyl cis-trans isomerase FKBP12 (MSC-FKBP12), and MSCs genetically engineered to express a fusion protein comprising the EV protein CD63 and FKBP12 (MSC-CD63FKBP12) isomerase, were cultured in MSCGM growth medium. To endogenously load MSC-EVs with rapamycin, a molecule directly binding FKBP12, MSC culture medium was replaced with Opti-MEM medium and incubated with 20 nM rapamycin for 12 h. Thereafter, the conditioned medium was collected and rapamycin-EVs isolated via tangential flow filtration and size exclusion chromatography. To exogenously load MSC-EVs with rapamycin, EVs from untreated MSC-FKBP12 and MSC-CD63FKBP21 cells were isolated using as above, then incubated with 500 nM rapamycin for 2 h, and re-isolated to remove rapamycin molecules that had not been loaded to EVs. Rapamycin loading to EVs is facilitated by its interaction of EV-loaded FKBP12 isomerase which leads to its “precharging” prior to its delivery to recipient cells, upon which FKBP-rapamycin complex inhibits mTOR activity.

The activity of EVs loaded exogenously or endogenously with rapamycin was tested in MCF7 breast cancer cells. MCF7 cells, cultured in DMEM/F12 medium supplemented with 10% FBS and antibiotics, were seeded to 24-well tissue culture plates. The next day, MCF7 cells were treated with rapamycin loaded EVs, naked rapamycin, non-loaded EVs, or left untreated. 24 hours after treatment, MCF7 cells were harvested and assayed for mTORC activity using the K-LIS mTOR Activity Kit (Merck Millipore).

### Example 5: Adrenomedullin Loading to EVs for Inducing Lipolysis in Adipose Cells

MSCs genetically engineered to express adrenomedullin (AM) CALCRL (a receptor for AM) as the binding protein or a binding protein specifically targeted to EVs using a CALCRL-CD81 fusion protein, were cultured in MSCGM growth medium, leading to endogenous loading of AM to secreted EVs. To isolate AM loaded EVs, MSC culture medium was replaced with Opti-MEM medium and incubated 48 h. Thereafter, the conditioned medium was collected and AM loaded EVs isolated via tangential flow filtration and size exclusion chromatography. The lipolytic activity of AM loaded EVs was tested in murine 3T3-L1 cells. 3T3-L1 cells, cultured in DMEM medium supplemented with 10% FBS and antibiotics, were seeded in 12-well tissue culture plates and subjected to differentiation, serum starved for 12 h and treated with free AM, AM loaded EVs, or controls. Relative change in glycerol release was measured using the free glycerol reagent (Sigma) according to manufacturer's recommendations.

1. An extracellular vesicle (EV) comprising a binding protein, characterized in that at least one small molecule agent is bound to the binding protein.

2. The EV according to claim 1, wherein the binding protein is selected from the group comprising GPCRs, polyclonal and monoclonal antibodies, single chain variable fragments (scFv), lipoproteins, integrins, tyrosine kinases, DNA-binding proteins, RNA-binding proteins, nucleases, ligases, proteases, integrases, isomerases, phosphatases, GTPases, aromatases, esterases, adaptor proteins, G-proteins, GEFs, cytokines, interleukins and interleukin receptor, interferons and interferon receptors, caspases, transcription factors, neurotrophic factors and their receptors, growth factors and their receptors, signal recognition particle and receptor components, extracellular matrix proteins, integral components of membrane, ribosomal proteins, translation elongation factors, translation initiation factors, GPI-anchored proteins, tissue factor, dystrophin, utrophin, dystrobrevin, and any fusions, combinations, subunits, derivatives, or domains thereof.

3. The EV according to any one of the preceding claims, wherein the at least one small molecule agent is a drug selected from the group comprising anticancer agents, cytostatic agents, DNA or RNA intercalators, nucleosides, splicing modulators, kinase inhibitors, tyrosine kinase inhibitors such as BTK inhibitors or Bcr-Abl inhibitors, statins, NSAIDs, antibiotics, antifungals, antibacterials, anti-inflammatory agents, anti-fibrotic agents, antihypertensives, vasodilators, hormones and analogues thereof, aromatase inhibitors, esterase inhibitors, anticholinergic agents, SSRIs, BKT inhibitors, PPAR agonists, dopamine agonists, HER inhibitors, AKT inhibitors, BCR-ABL inhibitors, signal transduction inhibitors, cytokine inhibitors, ILs and ILR inhibitors, TNF and TNFR inhibitors, angiogenesis inhibitors, synthase inhibitors, oligonucleotides such as siRNA, mRNA, antisense oligonucleotides, splice-switching oligonucleotides, peptides, cell-penetrating peptides, ALK inhibitors, BRAF inhibitors, MEK inhibitors, PI3K inhibitors, neprilysin inhibitors, beta2-agonists, CRTH2 antagonists, FXR agonists, BACE inhibitors, sphingosine-1-phosphate receptor modulators, MAPK inhibitors, Hedgehog signaling inhibitors, MDM2 antagonists, LSD1 inhibitors, lactamase inhibitors, IDO inhibitors, ERK inhibitors, Chk1 inhibitors, and any derivatives, subunits, domains, or combinations thereof.

4. The EV according to any one of the preceding claims, wherein the binding of the at least one small molecule agent optionally makes the binding protein therapeutically active.

5. The EV according to claim 4, wherein the therapeutically activated binding protein is signaling-competent or signaling-incompetent.

6. The EV according to any one of the preceding claims, wherein the binding protein is a fusion protein comprising a binding protein portion and an EV protein.

7. The EV according to claim 6, wherein the EV protein is selected from the group comprising CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, interleukin receptors, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof.

8. The EV according to any one of the preceding claims, further comprising a targeting moiety.

9. A method of exogenously producing EVs according to any one of claims 1 to 8, comprising the steps of:
a. providing EVs comprising a binding protein, optionally in the form of a fusion protein with an EV protein; and,
b. exposing the EVs to a small molecule agent to enable binding of the small molecule agent to the binding protein.

10. A method of endogenously producing EVs according to any one of claims 1 to 8, comprising the steps of:
a. incubating EV source cells comprising a binding protein, optionally in the form of a fusion protein with an EV protein, with at least one small molecule agent;
b. harvesting EVs produced by the EV source cells, wherein the EVs comprise the small molecule agent bound to the binding protein.

11. A method of delivering a binding protein-small molecule conjugate, a binding protein, and/or a small molecule agent, comprising the steps of:
a. providing an EV according to any one of claims 1 to 8; and,
b. delivering the EV to a target location.

12. The method according to claim 11, wherein the target location is a cell, a cellular or subcellular compartment, an organ, a tissue, and/or blood, bile, lymph, or interstitial fluids.

13. A pharmaceutical composition comprising EVs according to any one of claims 1 to 8 and a pharmaceutically acceptable carrier.

14. EVs according to any one of claims 1 to 8, or a pharmaceutical composition according to claim 13, for use in medicine.
